(19) United States
(12) Patent Application Publication
(10) Pub. No.: US 2026/0294396 A1
(43) Pub. Date: Oct. 1, 2026

(54) ULTRASOUND PROBE

(71) Applicant: FUJIFILM Corporation, Tokyo (JP)

(72) Inventors: Gen SHIINA, Tokyo (JP); Nobutaka MIYAMOTO, Tokyo (JP); Hitoshi TAKETOMO, Tokyo (JP); Kento UI, Tokyo (JP); Yudai SATO, Tokyo (JP)

(73) Assignee: FUJIFILM Corporation, Tokyo (JP)

(21) Appl. No.: 19/568,685

(22) Filed: Mar. 16, 2026

(30) Foreign Application Priority Data
Mar. 31, 2025 (JP) 2025-057474

(51) Int. Cl. A61B 8/00

(52) U.S. Cl. CPC A61B 8/4455

(57) ABSTRACT

An ultrasound probe includes: a head part that is long in a front-rear direction and incorporates a transducer array in which transducers that transmit and receive ultrasonic waves to and from a subject are arranged; and a grip part that has a rod shape and is connected to the head part. At least one finger holding groove is formed on one side surface of at least one of front, rear, left, or right side surface of the grip part. Each finger holding groove extends along a horizontal plane, a groove edge step is formed on a side edge of each finger holding groove, and no step is formed on an end edge of each finger holding groove.

### CROSS REFERENCE TO RELATED APPLICATION

This application claims priority to Japanese Patent Application No. 2025-057474 filed on Mar. 31, 2025 which is incorporated herein by reference in its entirety including the specification, claims, drawings, and abstract.

### BACKGROUND

### 1. Technical Field

The present disclosure relates to an ultrasound probe, and particularly to a shape of the ultrasound probe.

### 2. Description of the Related Art

An ultrasound probe that transmits and receives ultrasonic waves to and from a subject is known. An ultrasound image such as a tomographic image used for ultrasound diagnosis is formed based on the received ultrasonic waves. The ultrasound probe includes a head part and a grip part. A transducer array is built in the head part. An operator holds the grip part and brings the head part into contact with a diagnosis region of the subject. Then, a position and a posture of the ultrasound probe are changed by an operation such that an ultrasound image suitable for the diagnosis is obtained. Further, the ultrasound probe may be pressed against the subject depending on a situation.

Various shapes of the ultrasound probe have been proposed in consideration of a region of the subject that comes into contact with the head part and the operability of the operator, that is, ease of adjusting the position and the posture of the ultrasound probe. Further, the burden on a body of the operator such as the reduction in fatigue during long-term operation and the reduction in fatigue of fingers is also considered. In particular, the shape of the grip part is required to be suitable for changing the position and the posture of the ultrasound probe, for handling such as holding, pressing, and lifting, and for the grip in accordance with various ways of holding. Various shapes of the grip part have been proposed as described in JP6677049B, JP4616750B, Design Registration No. 1593697, and Design Registration No. 1591725

### SUMMARY

There is room for improvement in the shape of the ultrasound probe, particularly the shape of the grip part.

An aspect of the present disclosure relates to an ultrasound probe comprising: a head part that is long in a front-rear direction and incorporates a transducer array in which transducers that transmit and receive ultrasonic waves to and from a subject are arranged; and a grip part that has a rod shape and is connected to the head part. At least one finger holding part is formed on one side surface of at least one of front, rear, left, or right side surface of the grip part, the finger holding part has, at an edge on a head part side and on an edge on a side opposite to the head part side, stops on which a finger of an operator is stopped, and the finger of the operator is received between the stops. Each stop extends along a horizontal plane that is perpendicular to a plane defined by a longitudinal axis of the head part and a longitudinal axis of the grip part and is parallel to the longitudinal axis of the head part. Further, no stop on which the finger of the operator is stopped is provided at an end edge of the finger holding part in an extending direction of the stop.

In a case where the operator operates the ultrasound probe to press the ultrasound probe toward the subject and in a case where the operator operates the ultrasound probe to separate the ultrasound probe from the subject, the finger of the operator is stopped on the stop of the side edge of the finger holding part, and the slipping of the finger is suppressed. In a case where the operator rotates the grip part around the longitudinal axis, since the stop is not formed at the end edge of the finger holding part, the rotation operation is not hindered.

In the above-described ultrasound probe, the finger holding part may be provided on the left side surface and the right side surface of the grip part. The ultrasound probe can accommodate a holding manner in which the grip part is held by a thumb and one or a plurality of fingers facing the thumb.

In the above-described ultrasound probe, the finger holding part may be provided on the front side surface in addition to the left side surface and the right side surface of the grip part. The ultrasound probe can accommodate various holding manners.

In the above-described ultrasound probe, a bottom surface of the finger holding part, which is a surface between the stops, may be flat in a direction orthogonal to the horizontal plane. The operator can hold the ultrasound probe with the same sensation regardless of the position in the direction orthogonal to the horizontal plane in the finger holding part on which the finger is placed.

In the above-described ultrasound probe, the at least one finger holding part formed on one side surface may be a plurality of finger holding parts arranged along a longitudinal direction of the grip part. By providing the plurality of finger holding parts, various holding manners can be accommodated, and the slipping of the finger or the palm can be stopped at a plurality of locations.

In the above-described ultrasound probe, the plurality of finger holding parts may be arranged with an interval between adjacent finger holding parts.

In the above-described ultrasound probe, the finger holding part may have a groove shape extending along the horizontal plane, and may be a groove edge step in which the stop is formed at an edge of the groove shape.

In the above-described ultrasound probe, a curvature radius of a surface of the grip part in the horizontal plane may be larger at the bottom surface of the finger holding part than at a portion adjacent to the finger holding part. The groove is formed by a difference in the curvature radius. The bottom surface of the finger holding part may be a plane, and in this case, the orientation of the transducer array, that is, the front-rear direction is easily recognized from the feeling of holding the grip part.

In the above-described ultrasound probe, the grip part may be connected to a heel part of the head part and is disposed obliquely upward and rearward with respect to an arrangement direction of the transducers, and further, a step may be formed on the rear side surface of the grip part such that a portion of the grip part on the head part side from the finger holding part closest to the head part is thinner than a portion of the grip part on the other side from the finger holding part closest to the head part. Since the grip part is thin on the head part side, the position of the finger that comes into contact with the rear side surface of the grip part is stabilized, and the ultrasound probe can be firmly held.

In the above-described ultrasound probe, a concave portion curved rearward may be formed at a portion of the front side surface of the grip part adjacent to the head part. In a case of observing a flexion and extension state of the finger, a fingertip can be prevented from interfering with the grip part.

Another aspect of the present disclosure relates to an ultrasound probe comprising: a head part that is long in a front-rear direction and incorporates a transducer array in which transducers that transmit and receive ultrasonic waves to and from a subject are arranged; and a grip part that has a rod shape and is connected to the head part, in which at least one finger holding groove is formed on one side surface of at least one of front, rear, left, or right side surface of the grip part. Each finger holding groove extends along a horizontal plane that is perpendicular to a plane defined by a longitudinal axis of the head part and a longitudinal axis of the grip part and is parallel to an arrangement direction of the transducers. A step is formed on a side edge of each finger holding groove, and no step is formed on an end edge of each finger holding groove.

In a case of an operation of directing and pressing the ultrasound probe toward the diagnosis region of the subject and an operation of separating the ultrasound probe from the subject, the stop formed on the side edge of the finger holding part suppresses the slipping of the finger of the operator.

### BRIEF DESCRIPTION OF THE DRAWINGS

FIG. 1 is a perspective view of an ultrasound probe according to the present embodiment.

FIG. 2 is a side view of the ultrasound probe according to the present embodiment.

FIG. 3 is a front view of the ultrasound probe according to the present embodiment.

FIG. 4 is a view illustrating a finger holding groove provided in the ultrasound probe according to the present embodiment.

FIG. 5 is a horizontal cross-sectional view of a grip part of the ultrasound probe according to the present embodiment.

FIG. 6 is a vertical cross-sectional view of a finger holding groove part of the ultrasound probe according to the present embodiment.

FIG. 7 is a side view illustrating an ultrasound probe having a portion that is curved rearward at a portion of a front side surface of a grip part adjacent to a head part.

FIG. 8 is a rear view of the ultrasound probe in which the finger holding groove is formed on a rear side surface.

### DESCRIPTION OF EMBODIMENTS

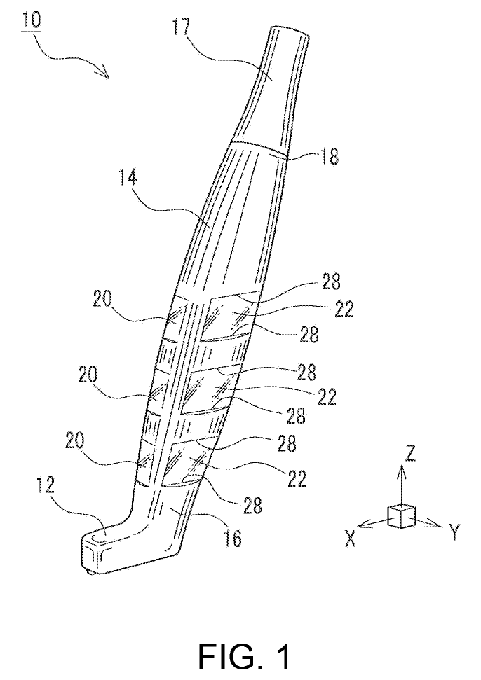

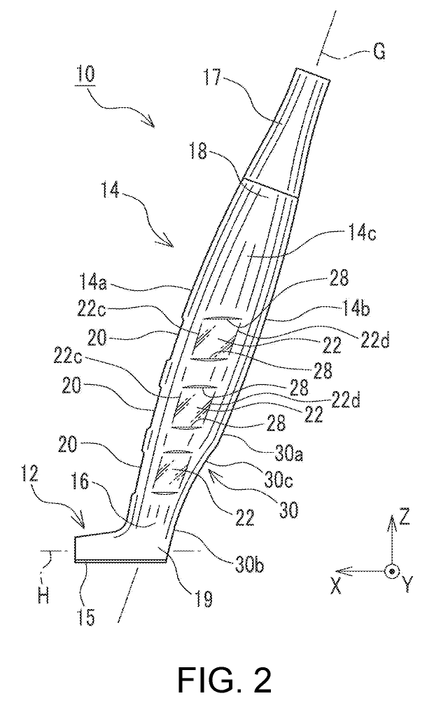

Hereinafter, embodiments of the present disclosure will be described with reference to the drawings. FIG. 1 is a perspective view illustrating a schematic shape of an ultrasound probe 10 according to the present embodiment. The ultrasound probe 10 includes a head part 12 and a grip part 14. The head part 12 incorporates a transducer array 15 (see FIG. 2) in which ultrasound transducers are arranged. The grip part 14 is a part that is gripped by an operator during the operation, and has a substantially rod shape. An axis along the longitudinal direction of the grip part 14 is referred to as an axis G (see FIG. 2). The head part 12 is connected to one end of the grip part 14. An end part of the grip part 14 on a side to which the head part 12 is connected is referred to as a hosel 16. The ultrasound probe 10 is connected to an ultrasound diagnostic apparatus (not illustrated) via a cable (not illustrated). A cable boot 17 is connected to an end of the grip part 14 opposite to the hosel 16. The end of the grip part 14 to which the cable boot 17 is connected is referred to as a grip end 18.

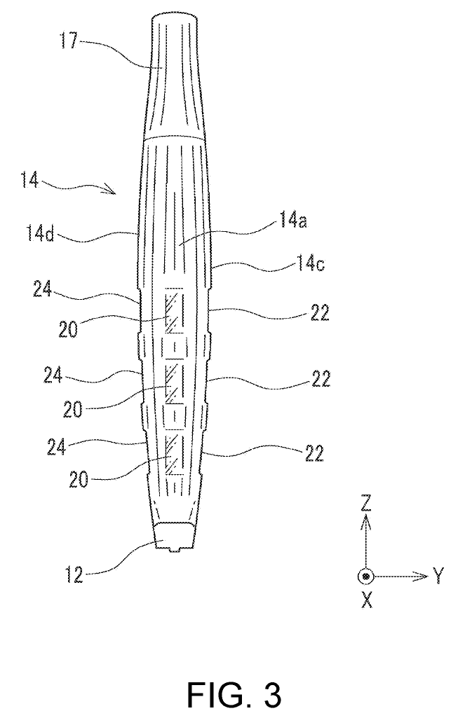

FIG. 2 is a side view illustrating a left side of the ultrasound probe 10, and FIG. 3 is a front view of the ultrasound probe 10. A right side surface of the ultrasound probe 10 is symmetrical to the side surface illustrated in FIG. 2.

An azimuth direction AZ that is an arrangement direction of a large number of ultrasound transducers constituting the transducer array 15 is a left-right direction in FIG. 2, and a direction along this direction is defined as an X-axis direction of a three-dimensional orthogonal coordinate system. A positive direction (a direction of an arrow X in FIG. 2) of the X-axis is a front direction, and a negative direction thereof is a rear direction. Among directions orthogonal to the azimuth direction (X-axis direction), a direction in a paper plane of FIG. 2 is a Z-axis direction, and a positive direction (a direction of an arrow Z in FIG. 2) of the Z-axis is an up direction, and a negative direction is a down direction. Further, among the directions orthogonal to the azimuth direction (X-axis direction), a direction vertically penetrating the paper plane of FIG. 2 is a Y-axis direction, and a positive direction (a front direction of the paper plane of FIG. 2) of the Y-axis is referred to as a left direction, and a negative direction (a rear direction of the paper plane of FIG. 2) is referred to as a right direction. Further, a direction of the transducer array 15 orthogonal to the azimuth direction AZ is referred to as an elevation direction EL. In the following description, unless otherwise noted, the relative positional relationship, the direction, and the orientation follow the above definitions.

The longitudinal direction of the head part 12 may coincide with the X-axis direction. Hereinafter, the direction along the longitudinal direction of the head part 12 is referred to as a front-rear direction. The axis extending in the longitudinal direction of the head part 12 is referred to as an axis H. The head part 12 and the grip part 14 are disposed such that the axes G and H (longitudinal axes) intersect each other, and a plane defined by the two axes G and H, that is, a plane including the axes G and H is referred to as a midplane. The ultrasound probe 10 may have a shape that is symmetric to the midplane. Further, a plane orthogonal to the midplane and parallel to the axis H of the head part 12 is referred to as a horizontal plane. The midplane is orthogonal to the Y-axis, and the horizontal plane is orthogonal to the Z-axis.

The transducer array 15 is disposed on a lower surface of the head part 12. The transducer array 15 may include an acoustic lens, and the acoustic lens may be a portion of a housing that defines an outer shape of the head part 12. The transducer array 15 transmits an ultrasonic beam downward in FIG. 2. The transducer array 15 receives the ultrasonic waves reflected from within the subject.

The head part 12 is substantially cuboid, and an upper surface thereof may be inclined in the X-axis direction such that the head part 12 is slightly narrowed toward a distal end of the head part 12 as illustrated in FIG. 2. The grip part 14 is connected to a rear end part (hereinafter, referred to as a heel part 19) of the head part 12. The grip part 14 is disposed such that the axis G thereof is oblique upward and rearward with respect to the axis H of the head part 12. The axis G of the grip part 14 may be connected to the heel part 19 at an angle of 110° to 120° with respect to the axis H of the head part 12. In addition, the grip part 14 may be connected to the head part 12 such that a rear end surface of the grip part 14 is coplanar with a rear end surface of the heel part 19. Since the grip part 14 is disposed to be inclined rearward with respect to the heel part 19 of the head part 12, the ultrasound probe 10 has a shape of a stick used in a hockey game as a whole.

The side surfaces of the rod-shaped grip part 14, that is, the front side surface 14a, the rear side surface 14b, the left side surface 14c, and the right side surface 14d are curved to be convex outward in the horizontal plane. A groove extending along the horizontal plane is formed on each of the front side surface 14a, the left side surface 14c, and the right side surface 14d. These grooves are referred to as finger holding grooves in the following description because, in a case where the operator who operates the ultrasound probe 10 places a finger on the groove, the finger can be held in the groove. The finger holding grooves provided on the front side surface 14a, the left side surface 14c, and the right side surface 14d are respectively referred to as a front finger holding groove 20, a left finger holding groove 22, and a right finger holding groove 24. Furthermore, three finger holding grooves 20 are arranged on the side surface 14a along the longitudinal direction of the grip part 14. Three finger holding grooves 22 are arranged on side surfaces 14c along the longitudinal direction of the grip part 14. Three finger holding grooves 24 are arranged on the side surfaces 14d along the longitudinal direction of the grip part 14.

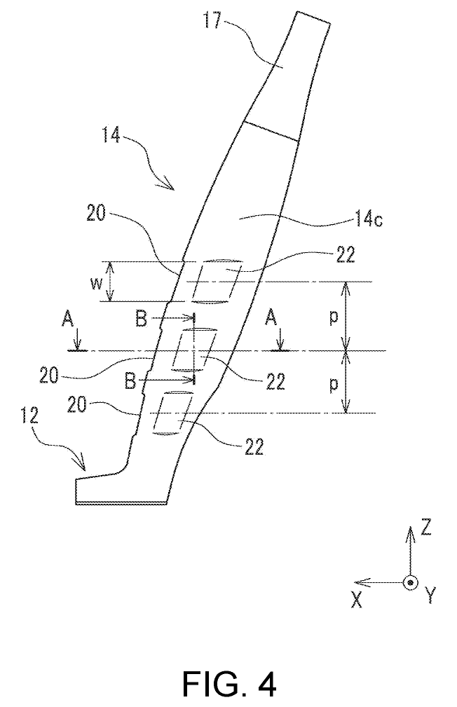

Each of the three left finger holding grooves 22 extends along the X-axis direction, and each width w (see FIG. 4) thereof may be 8 to 35 mm. Here, the width w of each of the finger holding grooves 20, 22, and 24 is a dimension in an up-down direction orthogonal to the direction in which the finger holding grooves extend. Further, an arrangement pitch p of the three left finger holding grooves may be 10 to 35 mm. The width w and the arrangement pitch p of the finger holding grooves 20, 22, and 24 are selected such that there is an interval between the adjacent finger holding grooves in a direction along the axis G of the grip part 14. The front finger holding groove 20 may have the same width w as the left finger holding groove 22, and may be disposed at the same position as the left finger holding groove 22 in the Z-axis direction. The right finger holding groove 24 may be formed symmetrically with the left finger holding groove 22 with respect to the midplane.

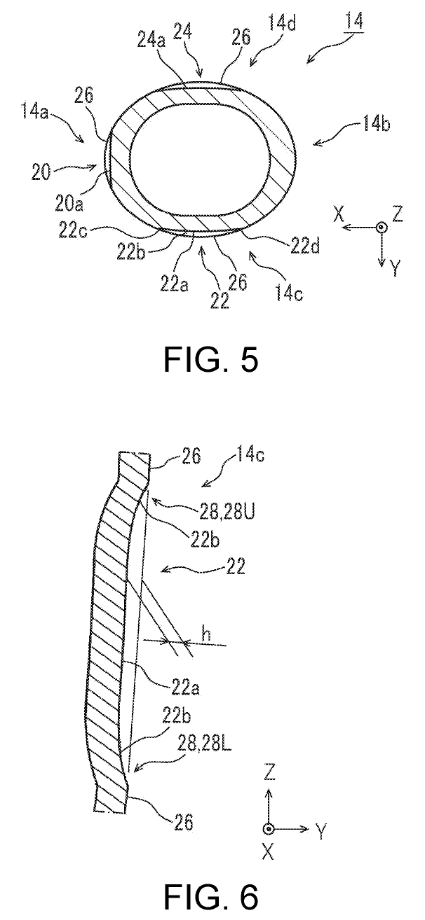

FIG. 5 is a cross-sectional view of the grip part 14 taken along line A-A in FIG. 4. The cross section illustrated in FIG. 5 is a cross-sectional view through the finger holding grooves 20, 22, and 24. FIG. 6 is a cross-sectional view of the left finger holding groove 22 and the grip part 14 around the left finger holding groove 22 taken along line B-B in FIG. 4. The vertical plane indicated by line B-B in FIG. 4 is a plane orthogonal to a direction in which the finger holding grooves 20, 22, and 24 extend. In FIGS. 5 and 6, an internal mechanism of the ultrasound probe 10 is not illustrated.

As illustrated in FIG. 5, the cross-sectional shape of the grip part 14 has a shape that is longer in the X-axis direction than in the Y-axis direction. Further, adjacent side surfaces among the four side surfaces 14a to 14d are connected by a curved surface and are smoothly connected without corners. The cross-sectional shape of the grip part 14 may be an ellipse or oval shape. Here, the ellipse or oval shape is not limited to the ellipse defined mathematically, and includes a shape obtained by deforming a circle in one direction. As described above, the finger holding groove is formed on the side surface of the grip part 14. A surface of the grip part 14 other than the finger holding groove part is referred to as a general surface 26.

The shape of each finger holding groove will be described by using the left finger holding groove 22 as an example. For convenience of notation, the left finger holding groove 22 will be simply referred to as the finger holding groove 22. The front finger holding groove 20 and the right finger holding groove 24 have the same configuration as the left finger holding groove 22.

The finger holding groove 22 forms a recess in Z-axis direction that is concave relative to the general surface 26 (see FIG. 6). A bottom surface 22a of the finger holding groove 22 is positioned at a low position (that is, a position inside the grip part 14) from the general surface 26 of a portion adjacent to the groove 22. A step is formed between the general surface 26 and an edge part of the finger holding groove 22 on the hosel 16 side and an edge part on the grip end 18 side. This step is referred to as a groove edge step 28, and in a case where it is necessary to distinguish between the step on the hosel side and the step on the grip end side, the step is referred to as a hosel side groove edge step 28L and a grip end side groove edge step 28U. The groove edge step 28 is formed by setting the general surface 26 as a higher surface, and the bottom surface 22a of the finger holding groove as a lower surface. A wall surface 22b that connects the higher surface (general surface 26) and the lower surface (bottom surface 22a) is provided between the higher surface and the lower surface. The wall surface 22b may be inclined. Further, the wall surface 22b may be a recessed curved surface, and may be smoothly connected to the bottom surface 22a. A depth h of the finger holding groove 22 may be 0.3 to 0.4 mm at the deepest point.

The bottom surface 22a of the finger holding groove may be curved convexly outward in the horizontal plane. The curvature radius of the bottom surface 22a of the finger holding groove in the horizontal plane is larger than the curvature radius of the general surface 26 adjacent to the finger holding groove 22 in the up-down direction. Further, the bottom surface 22a of the finger holding groove may be a plane. A step is not formed at a front end edge 22c and a rear end edge 22d of the finger holding groove 22, and the bottom surface 22a is directly connected to the general surface 26 of the grip part (see FIG. 5). The line connecting the front ends of the hosel side groove edge step 28L and the grip end side groove edge step 28U is the front end edge 22c of the finger holding groove 22. The line connecting the rear ends of the hosel side groove edge step 28L and the grip end side groove edge step 28U is the rear end edge 22d of the finger holding groove 22.

In a case of operating the ultrasound probe 10, the operator can hold the ultrasound probe 10 by placing the finger on an appropriate finger holding groove 20, 22, or 24. In a case where the operator brings the ultrasound probe 10 into contact with the diagnosis region of the subject, the finger of the operator is stopped on the hosel side groove edge step (the hosel side groove edge step 28L in the left finger holding groove 22) in the finger holding grooves 20, 22, and 24 on which the operator places the finger, particularly on the wall surface 22b, the slipping of the finger is suppressed, and the finger is held in the finger holding groove 22. In addition, in a case where the operator further presses the ultrasound probe 10 into contact with the diagnosis region, the finger of the operator is stopped on the hosel side groove edge step 28L, the slipping of the finger is suppressed, and the finger is held in the finger holding groove 22. In addition, since the finger is stopped on the hosel side groove edge step 28L, a force of pressing the ultrasound probe 10 against the diagnosis region can be reliably transmitted to the ultrasound probe 10. In a case where the operator separates the ultrasound probe 10 from the diagnosis region of the subject, the finger of the operator is stopped on the grip end side groove edge step (the grip end side groove edge step 28U in the left finger holding groove 22) in the finger holding grooves 20, 22, and 24 on which the operator places the finger, particularly on the wall surface 22b, the slipping of the finger is suppressed, and the finger is held in the finger holding groove 22. In a case where the operator holds the ultrasound probe 10 with the palm and the fingers, the palm is stopped on the hosel side groove edge step 28L or the grip end side groove edge step 28U together with the finger, and the slipping of the finger and the palm is suppressed.

In a case where the operator rotates the ultrasound probe 10 around the axis G of the rod-shaped grip part 14, since the steps are not provided at both end edges of each of the finger holding grooves 20, 22, and 24 in a direction in which the groove extends, the finger or the palm can be smoothly rotated without being stopped on the step.

The finger holding grooves 20, 22, and 24 are one aspect of the finger holding part. The groove edge step 28 formed at the edge of the groove shape is a stop on which the finger of the operator is stopped in a case where the operator operates the ultrasound probe 10. The finger of the operator is received between the two stops. The aspect of the finger holding part is not limited to the groove shape. For example, the finger holding part may be composed of a pair of ridges that protrude from the surface of the grip part 14 and extend along the surface and the surface of the grip part 14 between the ridges. These ridges extend along the horizontal plane. The ridge corresponds to the stop. A top of the ridge may be rounded. The finger holding part may be an area surrounded by two stops and lines connecting both ends of the two stops. The two stops and the lines connecting the two ends of the two stops are edges that define the finger holding part.

As illustrated in FIG. 2, a step is provided on the rear side surface 14b of the grip part 14. The step is referred to as a rear surface step 30. The rear surface step 30 consists of a high surface 30a, a low surface 30b, and a transition surface 30c connecting the surfaces. The high surface 30a and the low surface 30b may be substantially parallel surfaces, and the transition surface 30c may be inclined relative to the high surface 30a and the low surface 30b. The transition surface 30c is positioned at substantially the same position as the finger holding groove 22 closest to the hosel 16 side in the longitudinal direction of the grip part 14. The high surface 30a is positioned on the grip end 18 side from a position of the middle finger holding groove 22 in the longitudinal direction of the grip part 14, and the low surface 30b is positioned on the head part 12 side from a position of the finger holding groove 22 on the hosel 16 side. The rear surface step 30 causes the grip part 14 to be thinner on the hosel side and thicker on the grip end side.

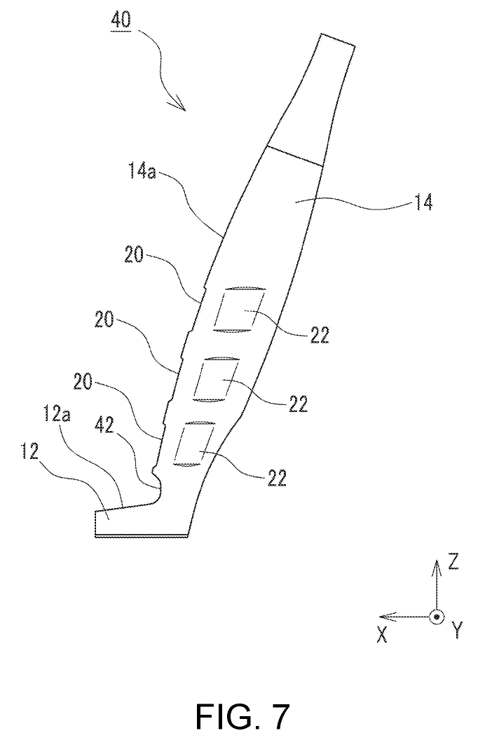

FIG. 7 is a side view illustrating an outer shape of an ultrasound probe 40 according to another embodiment. In the ultrasound probe 40, a concave portion 42 is formed in a region of the front side surface 14a of the grip part 14 on the hosel 16 side. The ultrasound probe 40 has the same configuration as the ultrasound probe 10 described above, except that the concave portion 42 is formed. The concave portion 42 is provided at a portion of the front side surface 14a of the grip part 14 adjacent to the head part 12 and is rearward. The concave portion 42 may be provided at a corner portion formed by the upper surface 12a of the head part 12 and the front side surface 14a of the grip part 14. In addition, the concave portion 42 may be formed rearward. The upper surface 12a of the head part 12 extends straight in the region of the concave portion 42, and a length of the upper surface 12a of the head part 12 in this case is longer than a length in a case where the concave portion 42 is not provided. For example, in a case where the subject's finger is diagnosed and the lower surface of the head part 12 is brought into contact with the pulp side and the position of the proximal phalanx of the finger of the subject and the state of flexion and extension of the finger of the subject is observed, the finger joint of the subject is bent such that the finger of the subject is wound around the distal end of the head part 12 while the head part 12 is brought into contact with the pulp side and the position of the proximal phalanx of the finger of the subject. In this case, the presence of the concave portion 42 prevents the fingertip of the subject from interfering with the grip part 14.

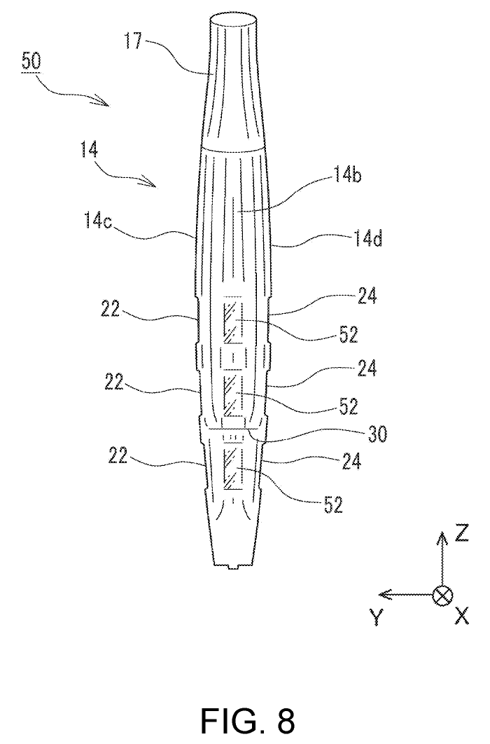

FIG. 8 is a rear view illustrating an ultrasound probe 50 according to still another embodiment. The ultrasound probe 50 includes the grip part 14 in which a rear finger holding groove 52 is formed on the rear side surface 14b. The configuration of the ultrasound probe 50 other than the rear finger holding groove 52 is the same as the configuration of the ultrasound probe 10 described above. Three rear finger holding grooves 52 are arranged on the rear side surface 14b of the grip part 14 at an interval between adjacent rear finger holding grooves 52 along the longitudinal direction of the grip part 14. A position of the rear finger holding groove 52 in the Z-axis direction may be the same as the positions of the other finger holding grooves 20, 22, and 24. The shape of each rear finger holding groove 52 may be the same as the shape of the left finger holding groove 22 described above, and the depth h and the groove width w of the rear finger holding groove 52 may be the same as those of the other finger holding grooves 20, 22, and 24.

In the ultrasound probes 10, 40, and 50 described above, three finger holding grooves are provided on one side surface, but the number of finger holding grooves is not limited to three. One, two, or four or more finger holding grooves may be provided on one side surface. The number of finger holding grooves provided for each side surface does not have to be the same.

In addition, the finger holding grooves formed in the grip part 14 may be formed on only one of the four side surfaces, only two of the four side surfaces, only three of the four side surfaces, or all of the four side surfaces. For example, the finger holding groove may be formed only on the front side surface 14a, or may be formed only on the left and right side surfaces 14c and 14d. In a case where the finger holding grooves are formed on a plurality of side surfaces, the width of the finger holding groove and the position in the Z-axis direction may be the same between the side surfaces.

What is claimed is:

1. An ultrasound probe comprising:
a head part that is long in a front-rear direction and incorporates a transducer array in which transducers that transmit and receive ultrasonic waves to and from a subject are arranged; and
a grip part that has a rod shape and is connected to the head part,
wherein at least one finger holding part is formed on one side surface of at least one of front, rear, left, or right side surface of the grip part, the finger holding part has, at an edge on a head part side and on an edge on a side opposite to the head part side, stops on which a finger of an operator is stopped, and the finger of the operator is received between the stops,
each stop extends along a horizontal plane that is perpendicular to a plane defined by a longitudinal axis of the head part and a longitudinal axis of the grip part and is parallel to the longitudinal axis of the head part, and
no stop on which the finger of the operator is stopped is provided at an end edge of the finger holding part in an extending direction of the stop.

2. The ultrasound probe according to claim 1,
wherein the finger holding part is provided on the left side surface and the right side surface of the grip part.

3. The ultrasound probe according to claim 2,
wherein the finger holding part is further provided on the front side surface of the grip part.

4. The ultrasound probe according to claim 1,
wherein a bottom surface of the finger holding part, which is a surface between the stops, is flat in a direction orthogonal to the horizontal plane.

5. The ultrasound probe according to claim 1,
wherein the at least one finger holding part formed on one side surface is a plurality of finger holding parts arranged along a longitudinal direction of the grip part.

6. The ultrasound probe according to claim 5,
wherein the plurality of finger holding parts are arranged with an interval between adjacent finger holding parts.

7. The ultrasound probe according to claim 6,
wherein the finger holding part has a groove shape extending along the horizontal plane, and is a groove edge step in which the stop is formed at an edge of the groove shape.

8. The ultrasound probe according to claim 7,
wherein a curvature radius of a surface of the grip part in the horizontal plane is larger at the bottom surface of the finger holding part than at a portion adjacent to the finger holding part.

9. The ultrasound probe according to claim 1,
wherein the grip part is connected to a heel part of the head part and is disposed obliquely upward and rearward with respect to an arrangement direction of the transducers, and
a step is formed on the rear side surface of the grip part such that a portion of the grip part on the head part side from the finger holding part closest to the head part is thinner than a portion of the grip part on the other side from the finger holding part closest to the head part.

10. The ultrasound probe according to claim 9,
wherein a concave portion curved rearward is formed at a portion of the front side surface of the grip part adjacent to the head part.

11. An ultrasound probe comprising:
a head part that is long in a front-rear direction and incorporates a transducer array in which transducers that transmit and receive ultrasonic waves to and from a subject are arranged; and
a grip part that has a rod shape and is connected to the head part,
wherein at least one finger holding groove is formed on one side surface of at least one of front, rear, left, or right side surface of the grip part, each finger holding groove extends along a horizontal plane that is perpendicular to a plane defined by a longitudinal axis of the head part and a longitudinal axis of the grip part and is parallel to the front-rear direction, a step is formed on a side edge of each finger holding groove, and no step is formed on an end edge of each finger holding groove.
